(19) United States
(12) Patent Application Publication
(10) Pub. No.: US 2004/0236302 A1
(43) Pub. Date: Nov. 25, 2004

(54) Wearable article having a humidity monitoring system

(76) Inventors: Hoa La Wilhelm, Appleton, WI (US); James Hongxue Wang, Appleton, WI (US)

Correspondence Address: Melanie I. Rauch, Pauley Petersen Kinne & Erickson, Suite 365, 2800 West Higgins Road, Hoffman Estates, IL, 60195, US

(21) Appl. No.: 10/442,004

(22) Filed: May 20, 2003

(51) Int. Cl. A61F 13/15

(52) U.S. Cl. 604/389

(57) ABSTRACT

A wearable article, such as an absorbent pant product, including a humidity monitoring system. A humidity sensor may be attached to a signal device that produces a signal when relative humidity inside the wearable article reaches a set level, such as 80%. A delivery device may also be connected to the humidity sensor, and may deliver a treatment to a wearer's skin when the relative humidity inside the absorbent article reaches the set level. The humidity monitoring system may be electromechanical, with the opening and closing of an electrical circuit being dependent on the level of relative humidity within the absorbent article.

### BACKGROUND OF THE INVENTION

This invention is directed to a wearable article having a built-in humidity monitoring system.

The primary function of wearable articles is typically to cover a wearer's body. Certain articles provide additional functions, such as absorbing bodily exudates, or protecting the body from contact with harmful sources. Attempts have been made to provide certain articles with the capability to monitor the health of the wearer. For example, certain articles have been outfitted with means to analyze human waste in order to detect specific health conditions such as urinary tract infections or yeast infections. Other articles have been equipped with a fever monitoring device.

Studies have shown that when the relative humidity inside a diaper reaches 80% to 85% or higher, this results in increased skin hydration and may be an indication of other conditions as well, such as temperature, pH and CO₂ levels. These and other factors, such as the presence of feces, eventually lead to rash and other health issues. In diapers having moisture-impermeable outer covers, for example, a high relative humidity environment is maintained within the diaper when soiled. Such occlusive coverings allow only limited evaporation of liquid from the skin.

Excessive hydration of the skin in the diaper area is one of several major causes of the development of diaper dermatitis. Maceration of the skin results from the nearly continuous exposure to moisture from urine, feces and perspiration. The barrier properties of the epidermis are compromised with excessive hydration enabling easier penetration of irritants into the skin. The coefficient of friction is higher for wet skin than it is for dry skin, making wet skin more susceptible to abrasion damage. One publication (J. Invest. Dermatol. 1998 November; 111(5):858-63) showed relatively constant levels of coefficient of friction and abrasion damage with exposure of skin to increasing fluid loads. It suggests that even at low loads (50 ml of urine in a diaper) the maximum susceptibility to friction and abrasion can be obtained. This mechanical injury also renders the skin more permeable to irritants. The increased hydration and damaged skin provide an excellent environment for the proliferation of microbes.

Increased humidity levels may occur in absorbent garments as well as in other types of wearable articles. The cause of increased humidity in such garments is not limited to expelled urine, but may instead be brought on by perspiration, or breathing, for example. More particularly, increased humidity levels may occur in diapers and other personal care garments, as well as in medical garments, athletic garments, and workwear garments. If the wearer or caregiver is made aware of the humidity level, or at least informed of the onset of a potentially detrimental humidity level, the wearer or caregiver could then take appropriate measures to prevent skin damage.

There is thus a need or desire for a wearable article capable of indicating a detrimental level of relative humidity within the article.

### SUMMARY OF THE INVENTION

This invention is directed to a wearable article, such as an absorbent article, including a humidity monitoring system. The humidity monitoring system includes a humidity sensor that measures the level of relative humidity within the article.

In one embodiment, a signal device may be connected to the humidity sensor, directly or indirectly, and may produce a signal when relative humidity within the article reaches a set level, such as about 80% or 85%. The signal may be a visual signal, an audio signal, a tactile signal, or a combination of any of these, for example, which informs the wearer or a caretaker that action should be taken to reduce the humidity within the article.

In another embodiment, a delivery device may be connected to the humidity sensor, directly or indirectly, and may be activated when the relative humidity within the article reaches a set level, such as about 80% or above. When the delivery device is activated it can deliver a composition, such as a lotion, cream, ointment, oil, rash treatment, athlete's foot treatment, vitamin A&D lotion, medicine, deodorant, skin coolant, or a combination of any of these, to the environment within the article, to the article itself, or more particularly, to a wearer's skin.

Both a signal device and a delivery device may be connected to the humidity sensor. Optionally, a temperature sensor may be included in the article to measure the temperature within the article.

The humidity monitoring system may be an electromechanical system including a circuit that connects the humidity sensor, and optionally a signal device and/or a delivery device, to a battery. The circuit is open when relative humidity (RH) within the article is below a set level, and is closed when the relative humidity within the article is above the set level. When the circuit closes, the signal device may produce a signal, and/or the delivery device may be activated. Alternatively, the closed circuit may trigger a mechanism to reduce the RH in the environment to a more comfortable level such as by releasing hygroscopic agents or drying agents including desiccants, or activating a micro-dehumidifier, or opening up a vent on the garment, or a combination of any of these.

The humidity monitoring system is particularly suitable for use in occlusive garments such as absorbent pant garments including diapers, training pants, and incontinence products, as well as other types of personal care garments, medical garments, athletic garments, and industrial workwear garments. For example, the humidity monitoring system can be applied to diapers, training pants, swim wear, absorbent underpants, adult incontinence products, feminine hygiene products, nursing pads, underarm pads, wipes, protective medical gowns, surgical medical gowns, infection protective suits, bandages, caps, gloves, drapes, face masks, laboratory coats, coveralls, sweatbands, athletic socks, shoes, helmet liners, hard hat liners, fire protection suits, and sports bras.

With the foregoing in mind, it is a feature and advantage of the invention to provide a wearable article capable of indicating a detrimental level of relative humidity within the article.

### BRIEF DESCRIPTION OF THE DRAWINGS

These and other objects and features of this invention will be better understood from the following detailed description taken in conjunction with the drawings, wherein:

FIG. 1 is a schematic diagram of one embodiment of a humidity monitoring system that may be included in a wearable garment.

FIGS. 2a & 2b are schematic diagrams of one embodiment of a circuit that may be included in a humidity monitoring system.

FIGS. 3a & 3b are schematic diagrams of another embodiment of a circuit that may be included in a humidity monitoring system.

FIG. 4 is a perspective view of a wearable garment into which a humidity monitoring system may be incorporated.

FIG. 5 is a plan view of a wearable garment with a humidity monitoring system incorporated therein, with the garment in a stretched flat state and showing the body-contacting surface of the garment.

### DEFINITIONS

Within the context of this specification, each term or phrase below will include the following meaning or meanings.

“Attached” refers to the direct or indirect joining, adhering, connecting, bonding, or the like, of two elements. Two elements will be considered to be attached together when they are attached directly to one another or indirectly to one another, such as when each is directly attached to intermediate elements.

“Body-contacting surface,” with respect to a garment, refers to a surface of the garment that comes into contact with a wearer's body, or closer to a wearer's body than the surfaces of other layers of the garment, when the garment is worn.

“Connected” refers to the joining, adhering, bonding, attaching, or the like, of two elements. Two elements will be considered to be connected together when they are connected directly to one another or indirectly to one another, such as when each is directly connected to intermediate elements.

“Liquid-impermeable,” when used to describe a layer or laminate means that liquid such as urine will not pass through the layer or laminate under ordinary use conditions in a direction generally perpendicular to the plane of the layer or laminate at the point of liquid contact.

“Liquid-permeable,” refers to a layer or laminate that is not liquid-impermeable.

“Relative humidity” or “RH” is defined as the ratio of the water vapor pressure or water vapor content to the saturation vapor pressure or the maximum vapor content at the temperature of the air or gas. The saturation vapor pressure in the air varies with air temperature: the higher the temperature, the more water vapor it can hold. When saturated, the relative humidity in the air is 100% RH.

$$Relative Humidity = \frac{actual vapor density}{saturation vapor density} \times 100 \%$$

Relative Humidity=actual vapor density×100% saturation vapor density

“Wearable article” includes personal care garments, medical garments, athletic and workwear garments, and the like. The term “disposable garment” includes garments which are typically disposed of after 1-5 uses. The term “personal care garment” includes diapers, training pants, swim wear, absorbent underpants, adult incontinence products, feminine hygiene products, nursing pads, underarm pads, wipes, breathable-when-dry outer absorbent product covers, shoes, and the like. The term “medical garment” includes medical (i.e., protective and/or surgical) gowns, infection protective suits, caps, gloves, drapes, face masks, bandages, and the like. The term “athletic garments” includes athletic socks, athletic shoes, pants, supporters, bras, shirts, sweat bands, helmet liners, and the like. The term “workwear garments” includes fire protective suits, laboratory coats, cover-alls, hard-hat liners, and the like.

These terms may be defined with additional language in the remaining portions of the specification.

### DESCRIPTION OF PREFERRED EMBODIMENTS

In accordance with the invention, a humidity monitoring system can be incorporated into a wearable article. As used herein, the term “wearable article” refers to a garment, accessory, or other article that can be adapted to fit about at least a portion of a wearer. Suitable articles to which the humidity monitoring system may be applied include personal care garments, medical garments, athletic garments, industrial workwear garments, and any other garments that contact the skin, directly or indirectly, and potentially cause increased humidity on the skin. More particularly, the humidity monitoring system may be applied to diapers, training pants, swim wear, absorbent underpants, adult incontinence products, feminine hygiene products, nursing pads, underarm pads, wipes, protective medical gowns, surgical medical gowns, infection protective suits, bandages, caps, gloves, drapes, face masks, laboratory coats, coveralls, sweatbands, athletic socks, shoes, helmet liners, fire protective suits, hard hat liners, and sports bras, for example.

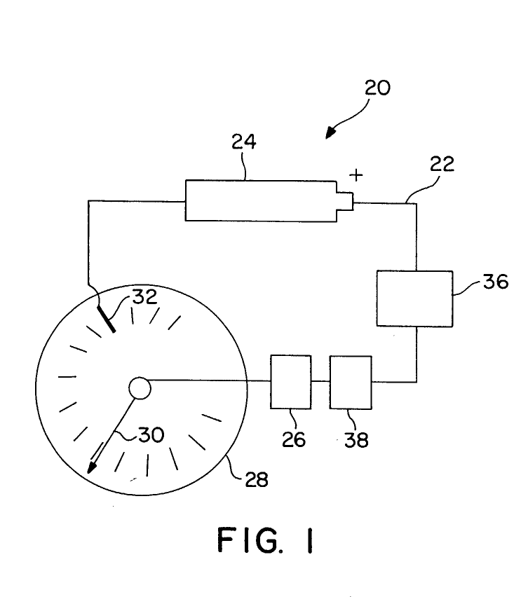

FIG. 1 is a diagram of one example of the humidity monitoring system 20. In this embodiment, the humidity monitoring system 20 is an electromechanical system with a circuit 22 connecting one or more components of the system to a power source 24, such as a miniature 3 volt watch battery or printed thin film lithium battery, or other suitable battery. Alternatively, the power source 24 may be a capacitor, solar cell, motion-induced battery, or combination of various types of power sources. One of the components is a humidity sensor 26, which measures the level of relative humidity (RH) within the article. The humidity sensor 26 may be connected to a humidity gauge 28. One example of a combined humidity sensor and humidity gauge is Dial Hygrometer/Thermometer 736920 available from Technika in Phoenix, Ariz. A threshold RH level can be set on the humidity gauge 28 and an RH needle 30 can be connected to a positive electrode of the power source 24, and a conductive bar 32 connected to a negative electrode of the power source 24 can be placed on the humidity gauge 28 at the threshold RH reading, such that below the set RH level the circuit 22 remains open. When the relative humidity within the article reaches the preset level, the circuit 22 closes. One example of a suitable threshold RH level is about 80%, or about 85%, since it has been found that skin irritation caused by skin hydration occurs when RH is at about 80% or about 85% in a closed environment such as in an absorbent garment.

The closed circuit may trigger one or more devices that either alert a caregiver or the wearer that action should be taken to prevent skin damage, or devices that administer a measure of relief. In one embodiment, for example, the humidity monitoring system 20 may include a signal device 36 connected to the humidity sensor 26. When the circuit 22 closes, the signal device 36 may produce one or more signals. Examples of suitable signals include visual signals, audio signals, tactile signals, and combinations thereof. Visual signals may exhibit a color change, fluorescence, or illumination, for example. Examples of color-indicating components include Gum Guaiac, or tetra-methyl-benzidine, or combinations thereof, which reacts when oxidized with an oxidizing component such as a peroxide or a per-acid or combinations thereof. Audio signals may exhibit an audible alarm or other noise. Although such devices may sound prohibitively expensive, particularly for use in disposable articles, suitable cost-efficient signal devices are readily available, such as the sound chips that are sometimes incorporated into greeting cards that play a tune or other noise when the greeting card is opened. Tactile signals may exhibit a temperature change sensation or other tactile change sensation on a surface of the article or the wearer's skin. In any case, the signal may be durable (i.e., stable and readable over a length of time, typically at least of the same magnitude as the usage life of the article) or transient (i.e., registering a real-time measurement).

In another embodiment, for example, the humidity monitoring system 20 may include a delivery device 38 connected to the humidity sensor 26. When the circuit 22 closes, i.e., when humidity within the article reaches the set level, the delivery device 38 may be activated. The delivery device 38 may include a pump or other mechanism that enables the delivery device 38 to release a treatment within the environment registering the set RH level. More particularly, the delivery device 38 may deliver the treatment to the wearer's skin or to the article. Examples of suitable treatments include lotions, creams, ointments, oils, rash treatment, athlete's foot treatment, vitamin A&D lotion, medicine, deodorant, skin coolant, and combinations thereof. Alternatively, the signal may trigger a mechanism to reduce the RH in the environment to a more comfortable level such as releasing hygroscopic agents or drying agents including desiccants, or activating a micro-dehumidifier, or opening up a vent on the garment. The delivery device may be adjustable to suit the wearer's specific needs and sensitivity level or treatment level desired.

In yet another embodiment, the humidity monitoring system 20 may include both a signal device 36 and a delivery device 38, thereby providing immediate relief to the wearer while simultaneously informing the wearer or the caregiver that the article should be removed and replaced.

In addition to the humidity monitoring system 20, a temperature sensor may also be attached to the wearable article, to provide further insight to the wearer or caregiver regarding the atmosphere within the wearable article. The temperature sensor may be connected to the humidity sensor 26, or may operate separately.

In another embodiment of the invention, the humidity sensor 26 may be chemically activated, rather than electro-mechanically activated. More particularly, the humidity sensor 26 may include, but is not limited to, any organic or inorganic, natural or synthetic materials such as polymers binders, superabsorbents, fibers, and the like, having a melting point or a phase transition at the predetermined threshold RH, or temperature and humidity combination, which is most likely to set off rash or skin irritation.

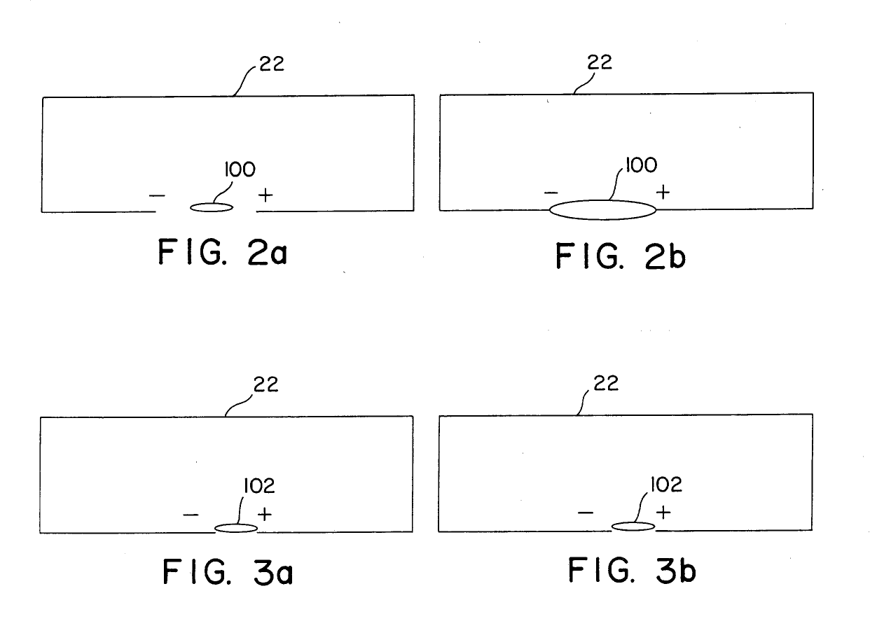

Another example of a circuit 22 is illustrated in FIGS. 2a and 2b. In this embodiment, a dry humidity sensitive agent 100, such as OASIS 101, available from Technical Absorbents, located in Grimsby, United Kingdom, is housed in a vapor/air-permeable and liquid/fluid-impermeable enclosure and placed between electrodes, as illustrated in FIG. 2a. When the humidity within the garment increases, the dry humidity sensitive agent 100 absorbs moisture from the environment or air inside the garment, forcing the dry humidity sensitive agent 100 to swell. More particularly, a single superabsorbent fiber can be used as the conductant element with its ends oriented toward the electrodes. When the humidity reaches the set level of RH, the dry humidity sensitive agent 100, or superabsorbent fiber, swells to such a degree that the agent 100 contacts the positive and negative electrodes, thereby closing the circuit 22, as illustrated in FIG. 2b. Any responses triggered by the closed circuit, such as the signal device and/or other delivery devices, will then be activated.

Yet another example of a circuit 22 is illustrated in FIGS. 3a and 3b. In this embodiment, a dry humidity sensitive agent 102, such as OASIS 101, available from Technical Absorbents, located in Grimsby, United Kingdom, is housed in a vapor/air-permeable and liquid/fluid-impermeable enclosure and placed in between and in contact with electrodes, as illustrated in FIG. 3a. As in the previous example, the agent 102 can be a single superabsorbent fiber. The agent 102 is non-conductant when dry and becomes a conductant upon sufficient wetting. The dry humidity sensitive agent 102 becomes sufficiently wet at the set level of RH and becomes a conductant, thereby closing the circuit 22, as illustrated in FIG. 3b. Any responses triggered by the closed circuit, such as the signal device and/or other delivery devices, will then be activated.

The humidity monitoring system 20, or at least the humidity sensor 26, can be housed inside a liquid-impermeable or one-way liquid-impermeable, air-permeable enclosure to prevent false signals resulting from exposure to urine, perspiration, or other liquids. The humidity monitoring system 20 can be located essentially anywhere in or on the article, as long as a humidity sensor 26 is not within the absorbent pad, if there is one in the garment, and is present within the environment of the garment around the skin area of concern. Suitably, the humidity sensor 26 is placed closest to the wearer's skin in the crotch portion of the body since the crotch region of the diaper or pants is often the wettest part of the product. As used herein, the term “sensor” refers to a component including one or more reactive means capable of detecting one or more target substances.

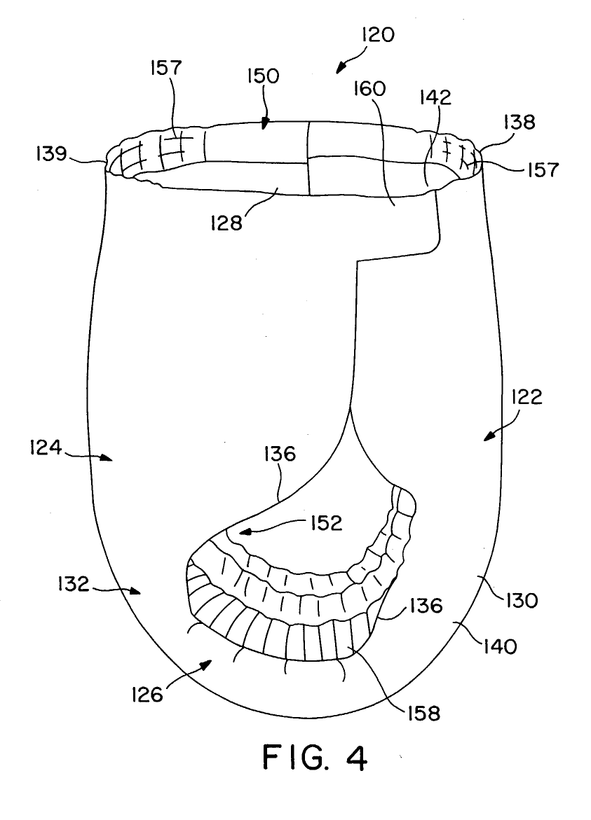

One example of a suitable absorbent pant garment, namely a diaper 120, into which the humidity monitoring system 20 may be incorporated is illustrated in FIG. 4. The diaper 120 includes a chassis 132. The chassis 132 defines a front region 122, a back region 124, a crotch region 126 interconnecting the front and back regions, a body-contacting surface 128 which is configured to contact the wearer, and an outer surface 130 opposite the body-contacting surface which is configured to contact the wearer's clothing. The front region 122 is contiguous with a front waist edge 138, and the back region 124 is contiguous with a back waist edge 139.

The diaper 120 includes an outer cover 140, a body side liner 142 which is connected to the outer cover in a superposed relation, an absorbent assembly (not shown) which is positioned or located between the outer cover 140 and the body side liner 142, and a pair of side panels 160 attached to the outer cover and/or the body side liner. These side panels 160 can be tabs, straps, tearable seams, or similar devices that can be fastened between the front region 122 and the back region 124 by suitable means, including adhesives.

As shown in the diaper 120 in FIG. 4, the front and back regions 122 and 124 together define a three-dimensional pant configuration having a waist opening 150 and a pair of leg openings 152. The waist edges 138 and 139 of the absorbent chassis 132 are configured to encircle the waist of the wearer when worn and provide the waist opening 150 which defines a waist perimeter dimension. Portions of the transversely opposed side edges 136 of the chassis 132 in the crotch region 126 generally define the leg openings 152. The front region 122 includes the portion of the diaper 120 which, when worn, is positioned on the front of the wearer while the back region 124 includes the portion of the diaper which, when worn, is positioned on the back of the wearer. The crotch region 126 of the diaper 120 includes the portion of the diaper which, when worn, is positioned between the legs of the wearer and covers the lower torso of the wearer. The side panels 160 of the diaper 120, when worn, are positioned forward from the hips of the wearer.

The chassis 132 is configured to contain and/or absorb any body exudates discharged from the wearer. For example, the absorbent chassis 132 can include a pair of elasticized containment flaps (not shown) which are configured to provide a barrier to the transverse flow of body exudates. The elasticized containment flaps define an unattached edge which assumes an upright, generally perpendicular configuration in at least the crotch region 126 of the training pant 120 to form a seal against the wearer's body. Suitable constructions and arrangements for the containment flaps are generally well known to those skilled in the art and are described in U.S. Pat. No. 4,704,116 issued Nov. 3, 1987 to Enloe, which is incorporated herein by reference.

To further enhance containment and/or absorption of body exudates, the diaper 120 can include waist elastic members 157 and/or leg elastic members 158, as are known to those skilled in the art (FIG. 4). The waist elastic members 157 can be operatively joined to the outer cover 140 and/or the body side liner 142 along the opposite waist edges 138 and 139, and can extend over part or all of the waist edges. The leg elastic members 158 are desirably operatively joined to the outer cover 140 and/or the body side liner 142 longitudinally along the opposite side edges 136 and positioned in the crotch region 126 of the diaper 120.

The outer cover 140 desirably includes a material that is substantially liquid-impermeable, and can be elastic, stretchable or nonstretchable. The outer cover 140 can be a single layer of liquid-impermeable material, but desirably includes a multi-layered laminate structure in which at least one of the layers is liquid-impermeable. For instance, the outer cover 140 can include a liquid-permeable outer layer and a liquid-impermeable inner layer that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid-permeable outer layer can be any suitable material and desirably one that provides a generally cloth-like texture. One example of such a material is a 20 gsm (grams per square meter) spunbond polypropylene nonwoven web. The outer layer may also be made of those materials of which liquid-permeable body side liner 142 is made. While it is not a necessity for the outer layer to be liquid-permeable, it is desired that it provides a relatively cloth-like texture to the wearer.

The inner layer of the outer cover 140 can be both liquid and vapor-impermeable, or can be liquid-impermeable and vapor-permeable. The inner layer is desirably manufactured from a thin plastic film, although other flexible liquid-impermeable materials may also be used. The inner layer, or the liquid-impermeable outer cover 140 when a single layer, prevents waste material from wetting articles, such as bedsheets and clothing, as well as the wearer and care giver. A suitable liquid-impermeable film for use as a liquid-impermeable inner layer, or a single layer liquid-impermeable outer cover 140, is a 0.2 millimeter polyethylene film commercially available from Huntsman Packaging of Newport News, Virginia, U.S.A. If the outer cover 140 is a single layer of material, it can be embossed and/or matte finished to provide a more cloth-like appearance. As earlier mentioned, the liquid-impermeable material can permit vapors to escape from the interior of the disposable absorbent article, while still preventing liquids from passing through the outer cover 140. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

The liquid-permeable body side liner 142 is illustrated as overlying the outer cover 140 and absorbent assembly, and may but need not have the same dimensions as the outer cover 140. The body side liner 142 is desirably compliant, soft feeling, and non-irritating to the wearer's skin. Further, the body side liner 142 can be less hydrophilic than the absorbent assembly, to present a relatively dry surface to the wearer and permit liquid to readily penetrate through its thickness.

The body side liner 142 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the body side liner 142. For example, the body side liner can be composed of a meltblown or spunbonded web of polyolefin fibers. The body side liner can also be a bonded-carded web composed of natural and/or synthetic fibers. The body side liner can be composed of a substantially hydrophobic material, and the hydrophobic material can, optionally, be treated with a surfactant or otherwise processed to impart a desired level of wettability and hydrophilicity.

The absorbent assembly can be positioned or located between the outer cover 140 and the body side liner 142, which components can be joined together by any suitable means, such as adhesives, as are well known in the art.

The absorbent assembly can be any structure which is generally compressible, conformable, non-irritating to the wearer's skin, and capable of absorbing and retaining liquids and certain body wastes. The absorbent assembly can be manufactured in a wide variety of sizes and shapes, and from a wide variety of liquid absorbent materials commonly used in the art. For example, the absorbent assembly can suitably include a matrix of hydrophilic fibers alone, such as a web of cellulosic fluff, or a mixture of both hydrophilic and hydrophobic fibers, or the fibers can be mixed with particles of a high-absorbency material commonly known as superabsorbent material. In a particular embodiment, the absorbent assembly includes a matrix of cellulosic fluff, such as wood pulp fluff, and superabsorbent hydrogel-forming particles. The wood pulp fluff can be exchanged with synthetic, polymeric, meltblown fibers or with a combination of meltblown fibers and natural fibers. The superabsorbent particles can be substantially homogeneously mixed with the hydrophilic fibers or can be nonuniformly mixed. The fluff and superabsorbent particles can also be selectively placed into desired zones of the absorbent assembly to better contain and absorb body exudates. The concentration of the superabsorbent particles can also vary through the thickness of the absorbent assembly. Alternatively, the absorbent assembly can include a laminate of fibrous webs and superabsorbent material or other suitable means of maintaining a superabsorbent material in a localized area.

Suitable superabsorbent materials can be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany. Typically, a superabsorbent material is capable of absorbing at least about 10 times its weight in water, and desirably is capable of absorbing more than about 25 times its weight in water.

The chassis 132 can also incorporate other materials that are designed primarily to receive, temporarily store, and/or transport liquid along the mutually facing surface with the absorbent assembly, thereby maximizing the overall absorbent capacity of the absorbent assembly, if desired. One suitable material is referred to as a surge layer (not shown) and includes a material having a basis weight of about 50 to about 120 grams per square meter (gsm), and including a through-air-bonded-carded web of a homogenous blend of 60 percent 3 denier type T-256 bicomponent fiber including a polyester core/polyethylene sheath and 40 percent 6 denier type T-295 polyester fiber, both commercially available from Kosa Corporation of Salisbury, N.C., U.S.A. Another example of a suitable surge layer may include a material made of 6 denier polyethylene terephthalate (PET) and 6 denier bicomponent binder fiber, having a basis weight of about 50 to about 120 gsm.

As described herein, the various components of the diaper 120 can be integrally assembled together employing various types of suitable attachment means, such as adhesive, ultrasonic and thermal bonds or combinations thereof.

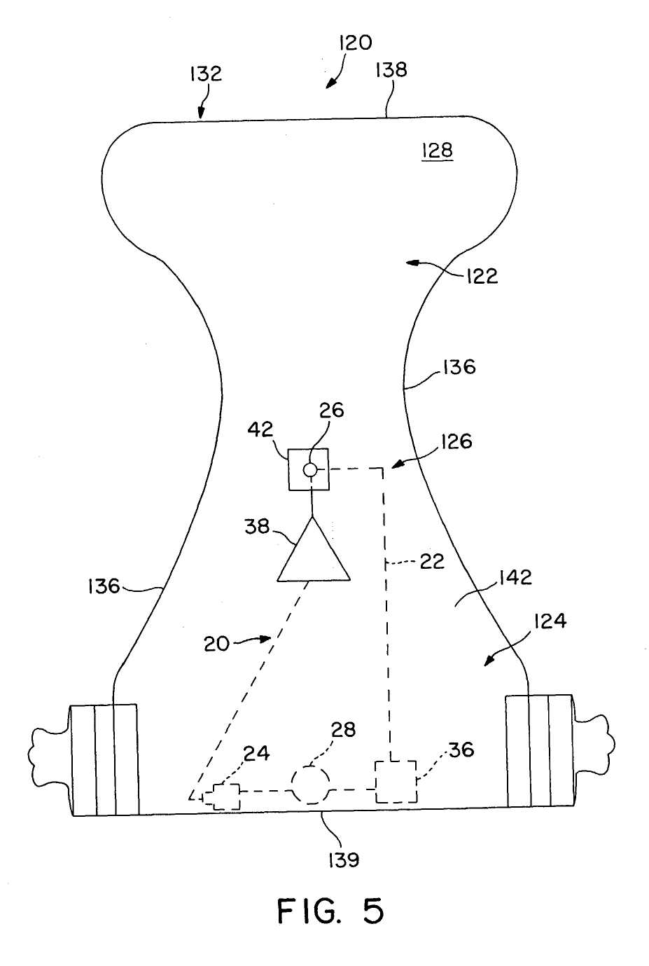

FIG. 5 illustrates a diaper 120 with the humidity monitoring system 20 incorporated therein. The humidity monitoring system 20 can be soldered or printed onto the body side liner 142 or other body-contacting surface 128 of the diaper 120. More particularly, the humidity sensor 26 can be located on the body side liner 142, or other body-contacting surface 128 of the diaper 120. Suitably, the humidity sensor 26 is located in a liquid-impermeable, air and/or vapor-permeable housing 42 placed close to a wearer's skin. Examples of suitable liquid-impermeable, air and/or vapor-permeable materials are described in greater detail above. The humidity sensor 26 inside the housing 42 can be located in any region of the diaper 120, such as the front region 122, back region 124, or crotch region 126, and is suitably located in any region least likely to cause discomfort to the wearer. The humidity sensor 26 can be included in a circuit, similar to the circuit 22 illustrated in FIG. 1. For example, the humidity sensor 26, and optionally the delivery device 38 (if present) can be attached to a body-contacting surface of the diaper 120, and the power source 24, humidity gauge 28, and signal device 36, all connected by the circuit 22, can be attached to the outer cover 140 or other outer surface 130 of the diaper 120, as illustrated in FIG. 5.

In one embodiment, the power source 24 is a solar cell, of the type used in solar calculators, for example, and is located on an outer surface of the garment. The circuit can be printed onto the garment and the whole system, including any responses, can be connected by soldering.

The humidity monitoring system 20 may include one or more components. An example of a one-component system is a humidity sensor 26 that includes a chemical, such as a polymer, that flows at 80% RH. The chemical may melt at a combination of RH and temperature. The chemical itself is triggered when the RH level, and possibly temperature, reaches the point of concern. Once triggered, the melting chemical may penetrate the one-way liquid-impermeable, air-permeable enclosure and react with at least one other chemical or at least one component of the urine or any component of the article or any combinations thereof to produce a visual, audio, or tactile signal to the wearer or caregiver that action should be taken to prevent skin damage. For example, in a diaper or other absorbent pant garment, the melting chemical may change the barrier tissue color from blue to red which can be seen through the transparent embossed window in the outer cover.

An example of a two-component humidity monitoring system includes a dye or an indicating component coated by a chemical or binder which flows or changes phase at a predetermined threshold RH, or RH and temperature combination, thereby releasing the dye to any part of the article as a visual signal.

Another example of a two-component humidity monitoring system includes a first chemical that changes its phase at a predetermined threshold temperature, and a second chemical that changes its phase at a predetermined humidity. A signal is produced only when the two chemicals react with each other.

Any combination of the humidity monitoring system components described here can be used without altering the scope of this invention.

While the embodiments of the invention disclosed herein are presently preferred, various modifications and improvements can be made without departing from the spirit and scope of the invention. The scope of the invention is indicated by the appended claims, and all changes that fall within the meaning and range of equivalents are intended to be embraced therein.

What is claimed is:

1. A wearable article comprising:
a substrate adapted to fit about at least a portion of a wearer; and
a humidity monitoring system including a humidity sensor attached to the substrate.

2. The wearable article of claim 1, wherein the humidity sensor is attached to a body-contacting surface of the substrate.

3. The wearable article of claim 1, wherein the humidity sensor is enclosed in a liquid-impermeable housing.

4. The wearable article of claim 1, wherein the humidity monitoring system further comprises a signal device connected to the humidity sensor, wherein the signal device produces at least one signal when relative humidity inside the wearable article reaches a set level or greater.

5. The wearable article of claim 4, wherein the at least one signal is selected from the group consisting of a visual signal, an audio signal, a tactile signal, and combinations thereof.

6. The wearable article of claim 4, wherein the set level of relative humidity is about 80%.

7. The wearable article of claim 1, wherein the humidity monitoring system further comprises a delivery device connected to the humidity sensor, wherein the delivery device is activated when relative humidity inside the wearable article reaches a set level or greater.

8. The wearable article of claim 7, wherein the delivery device delivers at least one of the group consisting of a lotion, a cream, an ointment, an oil, a rash treatment, athlete's foot treatment, vitamin A&D lotion, medicine, deodorant, skin coolant, and combinations thereof.

9. The wearable article of claim 7, wherein the delivery device reduces the relative humidity within the garment.

10. The wearable article of claim 9, wherein the delivery device uses at least one mechanism selected from the group consisting of releasing a hygroscopic agent, releasing a drying agent, activating a micro-dehumidifier, opening a vent on the garment, and combinations thereof.

11. The wearable article of claim 1, further comprising a temperature sensor attached to the substrate.

12. The wearable article of claim 1, wherein the wearable article is one selected from the group consisting of personal care garments, medical garments, athletic garments, and industrial workwear garments.

13. The wearable article of claim 12, wherein the wearable article is one selected from the group consisting of: diapers, training pants, swim wear, absorbent underpants, adult incontinence products, feminine hygiene products, nursing pads, underarm pads, wipes, protective medical gowns, surgical medical gowns, bandages, caps, gloves, drapes, face masks, laboratory coats, coveralls, infection protective suits, fire protective suits, sweatbands, athletic socks, shoes, helmet liners, hard hat liners, and sports bras.

14. A wearable article comprising:
a substrate adapted to fit about at least a portion of a wearer; and
an electromechanical humidity monitoring system including a sensor attached to the substrate.

15. The wearable article of claim 14, wherein the humidity sensor is attached to a body-contacting surface of the substrate.

16. The wearable article of claim 14, wherein the humidity sensor is enclosed in a liquid-impermeable housing.

17. The wearable article of claim 14, wherein the electromechanical humidity monitoring system comprises a circuit connecting a humidity sensor to a power source, and the circuit is open when relative humidity within the article is below a set level, and the circuit is closed when the relative humidity within the article is at or above the set level.

18. The wearable article of claim 17, wherein the set level of relative humidity is about 80%.

19. The wearable article of claim 17, wherein the electromechanical humidity monitoring system further comprises a signal device connected to the humidity sensor, wherein the signal device produces at least one signal when relative humidity inside the wearable article reaches the set level or greater.

20. The wearable article of claim 19, wherein the at least one signal is selected from the group consisting of a visual signal, an audio signal, a tactile signal, and combinations thereof.

21. The wearable article of claim 17, wherein the electromechanical humidity monitoring system further comprises a delivery device connected to the humidity sensor, and the delivery device delivers at least one of the group consisting of a lotion, a cream, an ointment, an oil, a rash treatment, athlete's foot treatment, vitamin A&D lotion, medicine, deodorant, skin coolant, and combinations thereof, when relative humidity within the garment reaches the set level.

22. The wearable article of claim 17, wherein the electromechanical humidity monitoring system further comprises a delivery device connected to the humidity sensor, and the delivery device reduces the relative humidity within the garment using at least one mechanism selected from the group consisting of releasing a hygroscopic agent, releasing a drying agent, activating a micro-dehumidifier, opening a vent on the garment, and combinations thereof.

23. An absorbent pant comprising:
a chassis defining a waist opening and first and second leg openings; and
a humidity monitoring system including a humidity sensor attached to the chassis.

24. The absorbent pant of claim 23, wherein the humidity sensor is attached to a body-contacting surface of the chassis.

25. The absorbent pant of claim 23, wherein the humidity sensor is enclosed in a liquid-impermeable housing.

26. The absorbent pant of claim 23, comprising an electrical circuit connecting the humidity sensor to a power source, wherein the circuit is open when relative humidity within the pant is below a set level, and the circuit is closed when the relative humidity within the pant is at or above the set level.

27. The absorbent pant of claim 26, wherein the set level of relative humidity is about 80%.

28. The absorbent pant of claim 23, wherein the humidity monitoring system further comprises a signal device connected to the humidity sensor, and the signal device produces at least one signal when relative humidity inside the absorbent pant reaches a set level or greater.

29. The absorbent pant of claim 28, wherein the at least one signal is selected from the group consisting of a visual signal, an audio signal, a tactile signal, and combinations thereof.

30. The absorbent pant of claim 23, wherein the humidity monitoring system further comprises a delivery device connected to the humidity sensor, and the delivery device delivers at least one of the group consisting of a lotion, a cream, an ointment, an oil, a rash treatment, athlete's foot treatment, vitamin A&D lotion, medicine, deodorant, skin coolant, and combinations thereof, when relative humidity within the absorbent pant reaches a set level or above.

31. The absorbent pant of claim 23, wherein the humidity monitoring system further comprises a delivery device connected to the humidity sensor, and the delivery device reduces the relative humidity within the garment using at least one mechanism selected from the group consisting of releasing a hygroscopic agent, releasing a drying agent, activating a micro-dehumidifier, opening a vent on the garment, and combinations thereof.
